(19) United States
(12) Patent Application Publication
(10) Pub. No.: US 2015/0274755 A1
(43) Pub. Date: Oct. 1, 2015

(54) COMPOUND DIVERSIFICATION USING LATE STAGE FUNCTIONALIZATION

(71) Applicants: Shane W. KRSKA, Rahway,, NJ (US); Petr VACHAL, Rahway,, NJ (US); Christopher J. WELCH, Rahway,, NJ (US); Graham F. SMITH, Boston,, MA (US); MERCK SHARP & DOHME CORP., Rahway,, NJ (US)

(72) Inventors: Shane W. Krska, New Providence, NJ (US); Petr Vachal, Summit, NJ (US); Christopher J. Welch, Cranbury, NJ (US); Graham F. Smith, Sudbury, MA (US)

(21) Appl. No.: 14/431,134

(22) PCT Filed: Sep. 20, 2013
(86) PCT No.: PCT/US13/60798

§ 371 Date: Mar. 25, 2015

Related U.S. Application Data
(60) Provisional application No. 61/705,350, filed on Sep. 25, 2012.

(51) Int. Cl. C07F 5/02; C07D 311/56

(52) U.S. Cl. CPC C07F 5/025; C07D 311/56

(57) ABSTRACT

The present invention relates to methods for generating limited chemical diversity for biologically relevant lead molecules by late state functionalization, separation, and post-functionalization modification. The methods optionally include one or more screening steps. Traditional drug development has relied on the synthesis of individual compounds or the generation of large chemical libraries, but these methods have generally been fairly inefficient in obtaining drug products.

### FIELD OF THE INVENTION

The present invention relates to methods for generating limited chemical diversity for lead drug candidate molecules by late stage functionalization, separation, and post-functionalization modification. The methods optionally include one or more screening steps.

### BACKGROUND OF THE INVENTION

Traditional drug development has relied on the synthesis of individual compounds or the generation of large chemical libraries, but these methods have generally been fairly inefficient in obtaining drug products. Oftentimes, drug candidates obtained by these methods are shelved late in development due to the emergence of liabilities such as metabolic stability, cell and membrane permeability, solubility and selectivity. Introducing limited chemical diversity into these failed drug candidates provides an opportunity to overcome these liabilities while gaining a better understanding of structural activity relationships (SAR). In this way, drug candidates can be optimized, providing a greater likelihood of success.

Late stage functionalization (LSF) is one means of introducing limited chemical diversity into a final product or advanced intermediate late in a synthetic sequence, as opposed to traditional approaches where incorporation of chemical diversity typically involves a change in starting materials,. One longstanding synthetic approach is known as “semisynthesis” in which complex molecular structures, usually derived from natural products, are chemically modified to introduce molecular diversity. See, e.g., Lv et al., 2011, Mini Rev. Med. Chem. 11:901-909; Altmann, et al., 2011, Molecular Diversity 15:383-399; and Gordaliza, 2007, Clinical and Translational Oncology, 9:767-776. Two main features distinguish LSF from semisynthesis: 1) in semisynthesis, new analogs are usually synthesized one at a time using traditional functional group manipulations, often requiring multiple synthetic steps per analog, while in LSF multiple analogs may be synthesized in one step using recently developed C—H functionalization chemistries; and 2) semisynthesis requires the presence of reactive functional groups (e.g., olefins, carbonyls, alcohols, amines, etc.) in the parent molecule, greatly limiting the potential sites of diversification, while LSF introduces diversity at ubiquitous C—H bonds which are inert to most traditional chemical methods.

The main advantage of LSF over traditional approaches is the efficiency with which multiple new chemical entities can be generated in one or two synthetic steps from an advanced intermediate or final product. Traditional approaches in most cases would require changing the starting materials, necessitating multiple synthetic steps to achieve each new desired analog. This increased efficiency can greatly reduce the cost and time requirements of making new analogs and open up new opportunities for SAR exploration that were heretofore cost prohibitive using traditional approaches.

In LSF, functionalization chemistries such as direct C—H functionalization allow straightforward modifications of lead drug candidates or advanced intermediates, giving chemists an ability to quickly spawn new versions of compounds wherein small changes with potentially improved activity or metabolic profile can be accessed quickly. Accordingly, interest in LSF is increasing due to its increasing utility as a tool for lead optimization. However, LSF has thus far been limited to generating site-specific modifications.

Nagib et al., 2011, Nature 480:224-228 describe a late-stage direct trifluoromethylation of arenes and heterarenes to protect against in vivo metabolism. No further modification is possible at the CF₃ group. Chen et al., 2009, Nature 459:824-828 describe parallel site-specific oxidation of C-H bonds and stepwise generation of multiple analogs. Dai et al., 2011, J Am Chem Soc 133:7222-7228 describe late-stage, site-specific diversification of a sulfonamide drug candidate containing multiple reactive C—H bonds. Additional lead diversification methods are described in Masood et al., 2012, Bioorg Med Chem Lett 22:723-728 and Massood et al., 2012, Bioorg Med Chem Lett 22:1255-1262.

The approaches described above generally only give one new analog per reaction and do not describe using the products of LSF as starting points for further diversification through post-functionalization modifications.

What is needed are new strategies for lead optimization which utilize simple chemistries while increasing diversity beyond that of site-specific LSF approaches.

### SUMMARY OF THE INVENTION

The present invention relates to methods for generating a compound library comprising the following steps: a) functionalizing a compound using a C—H functionalization chemistry to form a mixture of functionalized compounds; and b) performing post-functionalization modification chemistries on the C—H functionalized products to form a mixture of modified compounds. In certain aspects of the embodiment, the method provides for testing of one or more physical or biological properties of the compounds or mixture of compounds either after a) and before b), after b), or both.

In one embodiment, the mixture of functionalized compounds are separated prior to step b). Thus, in this embodiment, the method comprises a) functionalizing a compound using a C—H functionalization chemistry to form a mixture of functionalized compounds; b) separating the mixture of functionalized compounds to obtain individual compounds; and c) performing post-functionalization modification chemistries on the individual compounds. In certain aspects of this embodiment, the method provides for testing of one or more physical or biological properties of the compounds or mixture of compounds either after a) and before b), after b) and before c), after step c), or at all three points.

In another embodiment, the mixture of modified compounds are separated after step b). Thus, in this embodiment, the method comprises a) functionalizing a compound using a C—H functionalization chemistry to form a mixture of functionalized compounds; b) performing post-functionalization modification chemistries on the mixture of functionalized compounds to from a mixture of modified compounds; and c) separating the mixture of modified compounds to obtain individual compounds. In certain aspects of this embodiment, the method provides for testing of one or more physical or biological properties of the compounds or mixture of compounds either after a) and before b), after b) and before c), after step c), or at all three points.

The methods of the invention are generally applicable to any compound having C—H bonds. In certain embodiments, the compound is selected from heterocycles, steroids, alkaloids, and peptides/mimetics. In one aspect of the invention the compound has more than two or more functionalization sites. In one embodiment, the functionalizing step results in a mixture of mono- and poly-functionalized compounds. In certain embodiments, one or more functionalization sites are blocked with a protecting group.

Any C—H functionalization chemistry can be used in the methods of the invention. In certain embodiments, the functionalization chemistry is selected from C—H borylation, C—H halogenation (inclusive of all halogen elements from fluorine to iodine), C—H oxidation, C—H peroxidation, C—H acetoxylation, C—H amination, C—H arylation, C—H nitration, C—H carboxylation, C—H alkoxycarbonylation, C—H aminocarbonylation, C—H cyanation and C—H (fluoro)alkylation. C—H (fluoro)alkylation encompasses both C—H alkylation and C—H fluoroalkylation.

Separation is by any known means, preferably a method that separates individual components based on differences in molecular properties. In one embodiment, the separation is by a chromatography method. In another embodiment, mass spectroscopy is used to select chromatographic fractions for collection.

Modifying, i.e., performing one or more post-functionalization modification chemistries, can occur by any known chemistry for the particular functionalized group. In one embodiment, the modifying is selected from olefination, arylation, alkylation, borylation, halogenation, hydroxylation, cyanation, amination, alkoxylation, carboxylation, and carbonylation. In certain embodiments, the modifying is carried out in parallel.

The testing steps can be selected from any means for determining physical properties and/or biological properties. In certain embodiments, the testing can be selected from mass spectrometry, liquid chromatography, and NMR. In other embodiments, the testing is for a biological property.

Other embodiments, aspects and features of the present invention are either further described in or will be apparent from the ensuing description, examples and appended claims.

### DETAILED DESCRIPTION OF THE INVENTION

The present invention relates to methods for generating limited chemical diversity for biologically relevant lead candidate small molecules by late state functionalization, separation, and post-functionalization modification. The methods optionally include one or more screening steps to define appropriate reaction conditions for the late stage functionalization and post-functionalization modification steps and appropriate separation conditions for the separation step. These methods provide a limited number of chemical variations in lead candidates which can readily be screened for improved properties and optimization.

In traditional combinatorial chemistry, each reactant from a first group of reactants is reacted with each reactant from a second group of reactants to yield products containing all the combinations possible from the reaction. Additional reactions can be performed similarly to increase the size and diversity of the library. The result is a mixture of compounds which are, generally, iteratively screened as smaller mixtures.

In contrast, since, in the methods of the invention, application of C—H functionalization chemistries will result in mixtures of functionalized products, analysis and purification technologies are an important component of the LSF platform described herein. Application of LSF to one compound can lead to a mixture of several mono- and poly-functionalized derivatives, which can be subjected directly to biological testing and/or further elaborated into additional derivatives through downstream synthetic transformations on the newly installed functional handles, or can be separated to individual products prior to biological testing and/or further elaboration.

The LSF approach described herein can accelerate drug discovery efforts by: (1) Rapid SAR generation around leads from high-throughput screening of compound collections;

(2) Rapid improvement of physical or pharmacokinetic properties of “tool” compounds for target validation; (3) Installation of radiolabelled groups for target engagement and receptor occupancy studies as well as ADME studies; (4) Expansion of SAR around lead series in lead optimization space, giving greater IP coverage and improving physical and pharmacokinetic properties; (5) Transformation of inexpensive generic drugs into a library of high value analogs with new properties possibly providing proprietary leads starting from proven non-proprietary pharmacophores; and (6) Synthesis of known metabolites of drug candidates for analytical standards as well as for activity and ADME studies. Due to the chemistries utilized, a limited library of analogs can be produced in as little as two weeks. Preparation of a similar set of analogs using traditional chemistries can take months.

The LSF approach described herein may be illustrated as in Scheme 1.

A molecule of interest is subjected to any number of functionalization chemistry platforms, preferably a C—H functionalization chemistry, generating a mixture of mono- and polyfunetionalized products. Depending on the nature of the “X” group installed, this mixture of analogs may be subjected directly to chemical, physical or biological testing to determine if they have desirable properties, or be subjected to a preparative separation followed by determination of definitive structural identification of each isolated analog. For example, when X is OH, NRR′ (R, R′=alkyl, aryl or H), F, Cl, Br, CF₃, CO₂H, CN, alkyl, or aryl, the resulting compounds could be directly tested in biological assays. When X is, for example, boronate ester, Cl, Br, I, or OH, the resulting compounds from C—H functionalization (as a mixture or separated compounds) may be further chemically modified to generate families of derivatives from each functionalized analog. If post-functionalization is conducted directly on the product mixture from C—H functionalization, the resulting mixture may either be tested directly to identify compounds with desirable properties, or separated to give a library of individual functionalized analogs, each of which can then be tested for desirable properties.

This LSF approach can be used with any compound having C—H bonds. Classes of molecules, which are preferred focal points from which to obtain derivatives to serve as substrates, include heterocycles, steroids, alkaloids, and peptides/mimetics (including constrained molecules, e.g., constrained by S—S disulfide bonds). Examples of heterocycles include purines, pyrimidines, benzodiazepines, beta-lactams, tetracyclines, cephalosporins, and carbohydrates.

Examples of steroids include estrogens, androgens, cortisone, and ecdysone. Examples of alkaloids include ergots, vinca, curare, pyrollizidine, and mitomycines. Examples of peptides/mimetics include insulin, oxytocin, bradykinin, captopril, enalapril, and neurotoxins. A wide variety of drug analogs may be produced, such as analogs of antihypertensive agents, e.g. enalapril; beta-blockers, e.g. propanolol: antiulcer drugs (H₂-receptor antagonists) e.g. cirretidine and ranitidine; antifungal agents (cholesterol-demethylase inhibitors) e.g. isoconazole; anxiolytics, e.g. diazepam; analgesics, e.g. aspirin, phenacetamide, and fentanyl; antibiotics, e.g. vancomycin, penicillin and cephalosporin; antiinflammatories, e.g. cortisone; contraceptives, e.g. progestins; antihistamines, e.g. chlorphenamine; antitussives, e.g. codeine; sedatives, e.g. barbitol; etc. Preferred pharmacophores include benzodiazepines, beta-lactams, imidizoles and phenethylamines.

Preferably, the starting compound contains two or more functionalization sites in order to provide mixtures of functionalized compounds.

As used herein, the term “alkyl” refers to a monovalent straight or branched chain, saturated aliphatic hydrocarbon radical having a number of carbon atoms. For example, “alkyl” can refer to n-, iso-, sec- and t-butyl, n- and isopropyl, ethyl and methyl. Alkyl also encompasses saturated aliphatic hydrocarbon radicals wherein one or more hydrogens are replaced with deuterium, for example, CD₃.

The term “aryl” refers to phenyl, naphthyl, tetrahydronaphthyl, indenyl, dihydroindenyl and the like. An aryl of particular interest is phenyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms have been replaced with a halogen (i.e., F, Cl, Br and/or I). Thus, for example, “halomethyl” refers to a methyl group with one or more halogen substituents. The term “fluoroalkyl” has an analogous meaning except that the halogen substituents are restricted to fluoro. A fluoroalkyl of particular interest is CF₃.

The term “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I or its salt (e.g., the product isolated from a reaction mixture affording the compound or salt) consists of the compound or salt. The level of purity of the compounds and salts can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest level of purity governs. A compound or salt of 100% purity is one which is free of detectable impurities as determined by a standard method of analysis. It is understood that a substantially pure compound can be either a substantially pure mixture of stereoisomers or a substantially pure individual diastereomer or enantiomer.

### Functionalization

Functionalization of the starting compound is performed using, for example, a C—H functionalization chemistry under conditions which results in the generation of a mixture of mono- and poly-functionalized products, wherein the functional groups may be the same or different from each other. For example, in the case of Ir-catalyzed C—H borylation chemistry, all of the isomeric mono- and poly-functionalized products would contain the same newly installed functional group, namely a pinacolboronate ester or similar boronic acid derivative. Other functionalization chemistries that would result in the same functional group include direct C—H oxidation, C—H amination, C—H cyanation, C—H carboxylation, C—H alkoxycarbonylation, C—H aminocarbonylation, C—H di- and trifluoromethylation, C—H alkylation or C—H arylation. In the case of other functionalization chemistries such as metal-mediated halogenation reactions, it would be possible to generate mixtures of poly-functionalized compounds in which a mixture of halide and oxygen-containing (e.g., acetate, phenol, aliphatic alcohols, ketones) functional groups are present in the same molecule.

A functionalizable group may be blocked with a protecting group in order to ensure that only certain functionalization sites are functionalized during the reaction and/or to mask potentially interfering functional groups. A protecting group is any chemical group covalently bonded to a protected functionalization site group which prevents the functionalization site group from participating in the chemical reactions used to modify other functionalization sites. Protecting groups may include protecting groups traditionally used in the synthesis of peptides, such as t-butoxycarbonyl (BOC), or 9-fluorenylmethoxycarbonyl (Fmoc), benzyloxycarbonyl, 2-bromobenzyloxycarbonyl, 2-chlorobenzycarbonyl, 9-toluenesulfonyl, or mesitylene-2-sulfonyl. In addition, protecting groups may include a group within the same molecule to which the protected functionalization site group is covalently bonded, e.g., the activated acyl group in an anhydride acts as a protecting group for the other acyl group in an anhydride. The reaction should tolerate any number of protecting groups on nitrogen, as would be known to one of ordinary skill in the art, for example, BOC or Fmoc. More generally, any protecting group which does not interfere with reaction of the unprotected functionalization sites of the template may be utilized.

A protecting group may either become detached from the functionalization site group during the reaction of an unprotected functionalization site group, or the protecting group may be removed in a separate reaction prior to modification of the protected functionalization site group.

Examples of C—H functionalization chemistries for Step 1 include, but are not limited to, C—H borylation, C—H halogenation (inclusive of all halogen elements from fluorine to iodine), C—H oxidation, C—H peroxidation, C—H acetoxylation, C—H amination, C—H carboxylation, C—H alkoxycarbonation, C—H aminocarbonylation, C—H cyanation, C—H arylation, C—H nitration and C—H (fluoro)alkylation. See, e.g., Dai et al., 2011, J Am Chem Soc 133:7222-7228, Masood et al., 2012, Bioorg Med Chem Lett 22:723-728 and Massood et al., 2012, Bioorg Med Chem Lett 22:1255-1262. For a review, see Kakiuchi et al., 2003, Adv Synth Catal 345:1077. For each of these chemistries, a number of different reagents/catalysts/conditions are known. For example, there are numerous reports of Pd-catalyzed direct arylation, alkylation, halogenation, oxidation, and acetoxylation reactions of C—H bonds. In addition, Ir- and Rh-catalyzed C—H borylation reactions can be used. Finally, enzyme-catalyzed oxidations, such as those involving mammalian Cyp enzymes or cloned BM3 analogs, can also be used for the functionalization of aliphatic C—H bonds.

A wide range of efficient Ru- and Rh-catalyzed alkylations and arylations of aryl C—H bonds have been achieved with olefins or aryl organometallic reagents. See, e.g., Kakiuchi et al., 2003, J Am Chem Soc 125:1698; Lim et al., 2004, Org. Lett. 6:4687; Thalji et al., 2004, J Am Chem Soc 126:7192; Ackermann et al., 2006, Angew. Chem. Int. Ed. 45:2619. For Cu-catalyzed cross-dehydrogenation-coupling between sp³ and sp³ hybridized C—H bonds, see Li et al., 2005, J Am Chem Soc 127:3672. Pd-catalyzed alkenylation of aryl C—H bonds via Pd^{π}/Pd° catalysis has been reported. See Jia et al., 2001, Ace. Chem. Res. 34:633; and Boele et al., 2002, J Am Chem Soc 124:1586. Significant results have also been obtained using Ar₂I⁺X″ or ArX as the arylating reagents for sp² and sp³ hybridized C—H bonds involving Pd^{π}/Pd^{IV} catalysis. See Kalyani et al., 2005, J Am Chem Soc 127:7330; Daugulis et al., 2005, Angew. Chem. Int. Ed. 44:2; Shabashov et al., 2005, Org. Lett. 7:3657; Shabashov et al., 2005, J Am Chem Soc 127:13154. An alternative strategy involving C—H activation by an intramolecular ArPdX moiety has been developed. See Dyker, 1994, Angew. Chem. Int. Ed. 33:103; Catellani et al., 1997, Angew. Chem. Int. Ed. 36:119; Campo et al., 2003, J Am Chem Soc 125:11506; Campeau et al., 2004, J Am Chem Soc 126:9186; Bressy et al., 2005, J Am Chem Soc 127:13148; Dong et al., 2006, Angew. Chem. Int. Ed. 45:2289. In this context, another example of arylation of sp³ hybridized C—H bonds via Suzuki-Miyaura coupling has been achieved. See Barder et al., 2005, J Am Chem Soc 127:4685.

C—H oxidation by metalloporphyrin and metallosalen complexes is described in U.S. Pat. No. 6,002,026. C—H oxidation using palladium-catalyzed cross-coupling reaction is described in Wang et al., 2008, J. Am. Chem. Soc. 130:7190-7191. Additional C—H oxidation methods are taught in Chen et al., 2009, Nature 459:824-828; Chen et al., Science 2007; 318:783; and Chen et al., Science 2010, 327:566.

C—H halogenation using silver as a catalyst is described in Tang et al., 2010, J. Am. Chem. Soc. 132:12150-12154. C—H halogenation of aryls using palladium is described in Lee et al., 2011, Science 334:639-642.

C—H alkylation of 2-arylpryidine and arylpyrroles using organoboron reagents in the presence of a transition metal catalyst is described in International Patent Publication No. WO2008/024953. C—H fluoroalkylation of arene and heteroarenes using photoredox catalysis is described in Nagib et al., 2011, Nature 480:224-228. Additional C—H fluoroalkylation methods are taught in Ji et al., Proc. Nat. Acad. Sci. 2011; 108:14411-14415.

C—H amination of beta-lactam structures using a Lewis Acid co-catalyst is described in Qi et al., 2010, Tetrahedron 66:4816-4826. C—H amination of benzamides with aliphatic amines is described in Yoo et al., 2011, J. Am. Chem. Soc. 133:7652-7655. Amination of benzylic and unsubstituted aliphatic C—H bonds with aliphatic amines is described in Wiese et al., 2010, Angew. Chem. Int. Ed. 49:8850-8855. A similar transformation with aromatic amines is described in Gephart III et al., 2012, Angew. Chem. Int. Ed. 51:6488-6492.

The aminocarbonylation of C—H bonds of aromatic compounds to give benzolactams is described in Haffemeyer et al., 2011, Chem. Sci. 2:312-315 and López et al., 2011, Chem. Commun. 47:1054-1056. Aminocarbonylation of benzamides to give phthalimide derivatives is described in Inoue et al., 2009, J. Am. Chem. Soc. 131:6898-6899; Du et al., 2011, Chem. Commun. 47:12074-12076 and Wrigglesworth et al., 2011, Org. Lett. 13:5326-5329.

The alkoxycarbonylation of C—H bonds of benzylamine derivatives to give benzoate esters is described in Li et al., 2010, Dalton Trans. 39:10442-10446. C—H alkoxycarbonylation of phenyl-pyridines and -pyrazoles to give the corresponding benzoate esters is describes in Guan et al., 2009, J. Am. Chem. Soc. 131:729-733. Intramolecular C—H alkoxycarbonylation reactions yielding isochromanones is described in Lu et al., 2011, Chem. Sci. 2:967-971. C—H alkoxycarbonylation of indoles is described in Lang et al., 2012, Org. Lett. 14:4130-4133; Lang et al., 2011, Chem. Commun. 47:12553-12555 and Zhang et al., 2011, Chem. Eur. J. 17:9581-9585.

C—H carboxylation reactions are described in, for example, Itahara, 1982, Chem. Lett. 1151-1152; Giri and Yu, 2008, J. Am. Chem. Soc. 130:14082-14083 and Fujiwara et al., 1980 J. Chem. Soc., Chem. Commun. 220-221.

C—H cyanation reactions are described in, for example, Chen et al., 2006, J. Am. Chem. Soc. 128:6790-6791; Jia et al., 2009, Org. Lett. 11:4716-4719; Jia et al., 2009, J. Org. Chem. 74:9470-9474; Kim et al., 2010, J. Am. Chem. Soc. 132:10272-10274; Do et al., 2010, Org. Lett. 12:2517-2519; Reddy et al., 2010, Tetrahedron Lett. 51:3334-3336; Yan et al., 2010,

Org. Lett. 12:1052-1055; Ding et al., 2011, J. Am. Chem. Soc. 133:12374-12377; Ren et al., 2011, Chem. Commun. 47:6725-6727 and Kim et al., 2012, J. Am. Chem. Soc. 134:2528-2531.

C—H borylation reactions are described in, for example, Mkhalid et al., 2010, Chem. Rev.: 110:890-931; Liskey et al., 2012, J. Am. Chem. Soc. 134:12422-12425; Ros et al., 2011, Angew. Chem. Int. Ed. 50:11724-11728; Ishiyama et al., 2010, Chem. Commun. 46:159-161; Itoh et al., 2011, Chem. Lett. 40:1007-1008; Kawamorita et al., 2011, J. Am. Chem. Soc. 133:19310-19313; and Dai et al., 2012, J. Am. Chem. Soc. 134:134-137.

Since for each of the C—H functionalization chemistries given above, there are multiple sets of potential conditions, in order to efficiently assess which conditions give the best results for a particular substrate, parallel reaction screening techniques may be employed. Such screens can be conducted in microscale to limit the material requirements in cases where the substrate is in limited supply. Such parallel microscale screening techniques have been described in, for example, Shultz and Krska, 2007, Acc. Chem. Res., 40:1320-1326 and Preshlock et al., 2013, J. Amer. Chem. Soc. 135:7572-7582.

Most of the chemistries described above are amenable to post-functionalization modification. With C—H oxidation, amination, alkoxylation, (fluoro)alkylation, arylation, carboxylation and cyanation chemistry, post-functionalization modification is not required as the C—H functionalized products could be utilized directly in biological testing.

The chemistries described above are typically performed in solution, but can be performed using solid-phase chemistries. Various types of solid-phase chemistries are well-known in the art, and they may be used in conjunction with the various aspects of the invention. Examples of these solid-phase techniques include, but are not limited to, those described in Cho et al., 1993, Science 261: 1303-1305; DeWitt et al., 1993, Proc. Natl. Acad. Sci. U.S.A. 90:6909-6913; Simon et al., 1992, Proc. Natl. Acad. Sci. U.S.A. 89:9367-9371; Zuckermann et al., 1992, J. Am. Chem. Soc. 114:10646-10647; and Zuckermann et al., 1994, J. Med. Chem., 37: 2678-2685.

Solid-phase supports that can be used in a synthesis include materials such as polymers, resins, metals, glass beads, silica supports, gel or gel-type solid-phase supports (e.g., gel-type polystyrene resin solid-phase support, copolymer solid-phase supports (e.g., poly(styrene-oxyethylene graft copolymer supports), encapsulated gel solid-phase supports, macroporous supports, modified surfaces, and composite particles. A polymer that partly or substantially makes up a solid-phase support preferably includes polypropylene, polystyrene, chloroacetyl polystyrene, carboxypolystyrene, polystyrene-CHO, chloromethylated polystyrene polyamide, or polystyrene-poly(ethylene glycol) graft. Resins such as those made of imidazole carbonate resin, polyacrylamide resin, benzhydrol resin, p-nitrophenyl carbonate resin, diphenylmethanol resin, trityl alcohol resin, hydroxymethyl resin, or triphenyl methanol polystyrene resin, or their various combinations may also be used. Solid-phase supports such as resin beads and lanterns sold under the name SYNPHASE™ lanterns are commercially available.

Preferably, the dimension of the solid-phase support is no less than between about 0.6 mm to 0.7 mm, more preferably no less than about 0.5 mm. Polystyrene beads with dimensions ranging from approximately 500-600 μm are commercially available. The amount of solid-phase support placed in the wells of an array depends on factors such as the desired amount of products to be synthesized, as well as the well volume and the extent of swelling of the solid-phase support upon contact with a solvent or reagent. For example, a well may include between about 1-20 beads. Preferably, the synthesis produces at least about 1 mg of a reaction product, more preferably at least about 3-4 mg of a reaction product.

The type of solid-phase support to be used partly depends on one or more factors such as the desired amount of material to be synthesized (the loading capacity), compatibility of the chemistry intended for the library synthesis, and mode of attachment and cleavage of materials from the solid-phase support.

Preferably, a solid-phase support is prefunctionalized and may contain one or more functionalities or linkers. A solid-phase support may be functionalized with one or more chemically reactive groups that are used to attach a linker to a solid-phase support. Examples of these chemically reactive groups include, but are not limited to, isocyanates, carboxylic acids, esters, amides, alcohols, isothiocyanate, amines, and halomethyl groups. If desired, a solid-phase support that does not contain any functionalities or linkers may be used. Various non-functionalized solid-phase supports are commercially available such as certain types of SYNPHASE™ Lanterns.

Different types of linkers may be used in the various aspects of the invention. A linker covalently attaches molecules to the solid-phase support. The choice of a particular linker depends on factors such as the particular product or intermediate to be synthesized and the stability of a linker. Different types of linkers are known in the art and they are preferably attached to the solid-phase supports using standard solid-phase chemistry techniques. Preferably, the linkers are those based or adapted from protecting group chemistry.

Linkers that can be used with an array or method of the invention include acid labile linkers, nucleophile labile linkers, safety-catch linkers, traceless linkers, fluoride labile linkers, and photo-labile linkers. An advantage of nucleophile labile linkers is that it can be used to introduce a moiety or functional group during the cleavage step. Safety-catch linkers allow cleavage of an activated linker using mild conditions. Photo-labile linkers can be used under mild conditions and the process can be selective.

Other examples of linkers that may be used with an array or method of the invention include, but are not limited to, rink amide linkers, hydroxymethylphenoxy linker (HMP linker), backbone amide linker, trityl alcohol linker, disulfide linker, sulfoester linker, benzylhydryl or benzylamide linker, ortho-nitrobenzyl-based linker, nitroveratryoxycarbonyl-based linkers, and phenacyl based linkers.

Rink amide linkers, which are commercially available, can be used with activated carboxylic acids which cleave to form primary carboxyamides. Rink amide linkers can also be loaded with sulfonyl chloride to produce primary sulfonamides. When using rink amide linker, cleavage is normally performed using 20% trifluoroacetic acid (TFA)/dichloromethane (DCM). Solid-phase supports with HMP linker can be used to link carboxylic acids, phenols, and amines. In this case, cleavage via acidolysis produces the original functional group. The carboxylic acids can be coupled using N,N′-diisopropylcarbodiimide (DIC)/N,N-dimethylaminopyridine through imidate or Mitsunobu chemistry. With HMP linker, cleavage is normally performed with about 20% or higher concentrations of TFA/DCM. Trityl alcohol linker can be used to link carboxylic acids, alcohols, phenols, and amines. Cleavage via acidolysis produces the original functional group. Hyperlabile linker links phenols, amines, and carboxylic acids. Cleavage by acidolysis recovers the original functional group.

Cleavage of the synthesis products can be performed using techniques known in the art. In one aspect, a product is cleaved from the solid-phase support via an intramolecular reaction that removes the compound from the solid-phase support without leaving any trace of the site of attachment. See DeWitt et al., 1993, Proc. Natl. Acad. Sci. U.S.A. 90:6909-6913.

### Separation

After functionalization, the resulting mono- and poly-functionalized products may be separated using traditional technologies to obtain isolated compounds. Preferably, these technologies would employ mass spectrometry to select fractions for collection on the basis of mass, and these would allow the mono- and poly-functionalized products to be selectively isolated in the presence of other by-products and degradants that are not of interest.

Examples of separations technologies include, but are not limited to, normal phase (flash) chromatography, mass-directed preparative reversed phase chromatography, supercritical fluid chromatography, two-dimensional chromatography and preparative thin-layer chromatography.

### Post-Functionalization Modification

After separation, post-functionalization modification occurs to introduce a desired group at each of the functionalized moieties. A reactant containing a functional group is capable of reacting with a functionalized site on the functionalized compound. A reactant is any chemical which can undergo a chemical reaction to form a new bond. Because the functionalized sites, reactants and the reaction conditions are not limited, the functionalized compounds can be designed for use with a very broad spectrum of chemical reactions.

The reactants added to the functionalized sites provide molecular diversity. In some embodiments, one or more of the separated functionalized compounds may be set aside, undergoing no reaction.

Post functionalization modification reactions include, but are not limited to, olefination, arylation, alkylation, halogenation, amination, alkoxylation, cyanation, carboxylation, and carbonylation. See Dai et al., 2011, J Am Chem Soc 133:7222-7228. Examples of post-functionalization derivatization chemistries for Step 3 will depend on the nature of the group X that was installed in Step 1. For X=halogen on aliphatic positions, classical displacement or elimination reactions could be employed. For halogenated aromatic sites, metal-catalyzed cross-coupling chemistries such as Suzuki couplings with aryl and alkyl boronic acid derivatives, Negishi couplings with aryl and alkyl zinc reagents, Buchwald-Hartwig aminations with aromatic and aliphatic amines and amides, C—O, C—S and C—F couplings, and trifluoromethylations could be used. For X=OH on aliphatic positions, classical activation/displacement chemistries could be employed such as the Mitsunobu reaction. Oxidation to an aldehyde or ketone moiety could also be followed by reductive amination, addition reactions with carbon-based nucleophiles, or transformations to fluorine-bearing positions using reagents such as DEOxO-FLUOR™ (Scott Medical Products, Plumsteadville, Pa.). For aromatic OH groups, activation with a sulfonic acid derivative (such as trifluoromethylsulfonyl anhydride) followed by similar cross-coupling reactions as described above for halogenated aromatics could be employed. For X=boronic acid derivatives (e.g., pinacol boronate ester), a variety of cross-coupling reactions could be employed. Examples include: Suzuki coupling with aryl halides; Chan-Lam oxidative coupling reactions with amines, heterocycles, alcohols, thiols, and alkynes; oxidative coupling reactions with trifluoromethyl copper, silicon and silver reagents, transition-metal and photochemically catalyzed coupling with (fluoro)alkyl halides, coupling with trifluoromethoxy organometallics, with nucleophilic and electrophilic fluorinating or other halogenating reagents, oxidative carboxylation, carbonylation and cyanation reactions, and oxidation to phenols.

In order to find the optimum post-functionalization reaction conditions for each functionalized product, these reactions may be screened in parallel using microscale techniques described above. See, e.g., Preshlock et al., 2013, J. Amer. Chem. Soc. 135:7572-7582.

Generally, the methods of the invention provide for a limited diversification around the compound of interest. The methods are suitable for formation of 10-50, 10-100, 10-500 or 10-1000 compounds.

### Testing of Chemical, Physical and Biological Properties

To determine the characteristics of the analogs, a wide variety of assays and techniques may be employed. Such testing methods are well known to those skilled in the art and are typically employed in traditional drug development. Testing can occur after functionalization, after separation and/or post-functionalization modification. Assays are generally performed using individual compounds, but in some cases can be conducted on mixtures of compounds.

Various assay conditions may be used for the detection of binding activity as will be described subsequently.

In some instances, one may be able to carry out a two-stage screen, whereby one first uses, for example, binding as an initial screen, followed by biological activity with a viable cell in a second screen.

### Physical

Physical analysis can be performed to validate the reactions to ensure the correct product was formed and in good yield. The cleaved compounds may be analyzed or characterized using one or more analytical techniques such as mass spectrometry, liquid chromatography, NMR, MALDI-TOF mass spectrometry, or a combination of techniques such as LC-UV/MS.

Where solid-phase chemistries are used, on-site or on-bead analysis may be performed using techniques such as magic angle spinning NMR or FT-IR spectrometry. The applications of one or more of these techniques in solid-phase synthesis have been described in several publications including, for example, Chu et al., 1993, J. Org. Chem. 58:648-652; Fitch et al., 1994, J. Org. Chem. 59:7955-7956; Gao et al., 1996, J. Med. Chem. 39:1949-1955; Keifer, 1996, J. Org. Chem., 61: 1558-1559; Metzger et al., 1993, Angew. Chem. Int. Ed., 32: 894-896; Stevanovich et al., 1993, Anal. Biochem. 212:212-220; and Youngquist et al., 1994, Rapid Commun. Mass Spectrom. 8:77-81.

### Biological

Of particular interest is finding products that have biological activity. Analogs produced using the methods of the invention can be tested for improved properties such as efficacy, bioavailability, toxicity, etc. In some applications it is desirable to find a product that has an effect on living cells, such as inhibition of microbial growth, inhibition of viral growth, inhibition of gene expression or activation of gene expression. Screening of the compounds on the beads, if used, can be readily achieved, for example, by embedding the beads in a semisolid medium and the library of product molecules released from the beads (while the beads are retained) enabling the compounds to diffuse into the surrounding medium. The effects, such as plaques within a bacterial lawn, can be observed. Zones of growth inhibition or growth activation or effects on gene expression can then be visualized and the compound at the center of the zone picked and analyzed.

The libraries may be screened for compounds that bind to individual cellular receptors, or functional portions of the individual cellular receptor (and may additionally be capable of disrupting receptor function). The receptor may be a single molecule, a molecule associated with a microsome or cell, or the like. One such method for identifying an agent to be tested for an ability to bind to and potentially modulate a cellular receptor signal transduction pathway is as follows. The method involves exposing at least one compound from the libraries to a protein comprising a functional portion of a cellular receptor for a time sufficient to allow binding of the library compound to the functional portion of the cellular receptor; removing non-bound compound; and determining the presence of the compound bound to the functional portion of the cellular receptor, thereby identifying a compound to be tested for an ability to modulate a cellular receptor signal transduction pathway.

Various devices are available for detecting cellular response, such as a microphysiometer, available from Molecular Devices, Redwood City, Calif.. Where binding is of interest, one may use a labeled receptor, where the label is a fluorescer, enzyme, radioisotope, or the like, where one can detect the binding of the receptor to the compound on the bead. Alternatively, one may provide for an antibody to the receptor, where the antibody is labeled, which may allow for amplification of the signal and avoid changing the receptor of interest, which might affect its binding to the product of interest. Binding may also be determined by displacement of a ligand bound to the receptor, where the ligand is labeled with a detectable label.

As indicated above, cells can be genetically engineered so as to indicate when a signal has been transduced. There are many receptors for which the genes are known whose expression is activated. By inserting an exogenous gene into a site where the gene is under the transcriptional control of the promoter responsive to such receptor, an enzyme can be produced which provides a detectable signal, e.g. a fluorescent signal. The particle associated with the fluorescent cell(s) may then be analyzed for its reaction history.

One technique that is used for screening mixtures of compounds derived directly from Late Stage Functionalization without the need for an intervening purification/separation step is affinity selection mass spectrometry (AS-MS). This method determines relative protein-ligand affinity ranking, and can distinguish between allosteric and orthosteric binding modes. AS-MS consists of three stages: an affinity selection stage where compounds are selected based on their protein binding affinity relative to varying concentrations of a competitor ligand, a first chromatography stage that separates protein-ligand complexes from unbound ligands, and a second chromatography stage that induces dissociation of the protein-ligand complexes and identifies and quantifies the formerly bound ligands by mass spectrometry. This technique, and its application to drug discovery, has been described in numerous published reports, among them Annis et al., 2004, J. Am. Chem. Soc. 126:15495-15503; Annis et al., 2007, Curr. Opin. Chem. Biol. 11:518-526; and Huang et al., 2012, ACS Med. Chem. Lett. 3:123-128.

### Abbreviations

Abbreviations employed herein include the following:

BOC or Boc=t-butyloxycarbonyl

[(COD)IrOMe]2=bis[(μ-methoxy)(1,5-cyclooctadiene)iridium(I)] complex

CuBr₂=copper (II) bromide

CuCl₂=copper (II) chloride

DCM=dichloromethane;

Fmoc=9-fluorenylmethoxycarbonyl

Ir=iridium

Me=methyl

MeCN=acetonitrile

MS=mass spectrometry

NaIO₄=sodium periodate

NMR=nuclear magnetic resonance

Pd=palladium

Rh=rhodium

RT=room temperature

Ru=ruthenium

TFA=trifluoroacetic acid

THF=tetrahydrofuran

### EXAMPLE 1

All reagents were obtained from commercial sources unless otherwise noted. For a representative compound for the methods of the present invention, Warfarin is chosen for late stage functionalization.

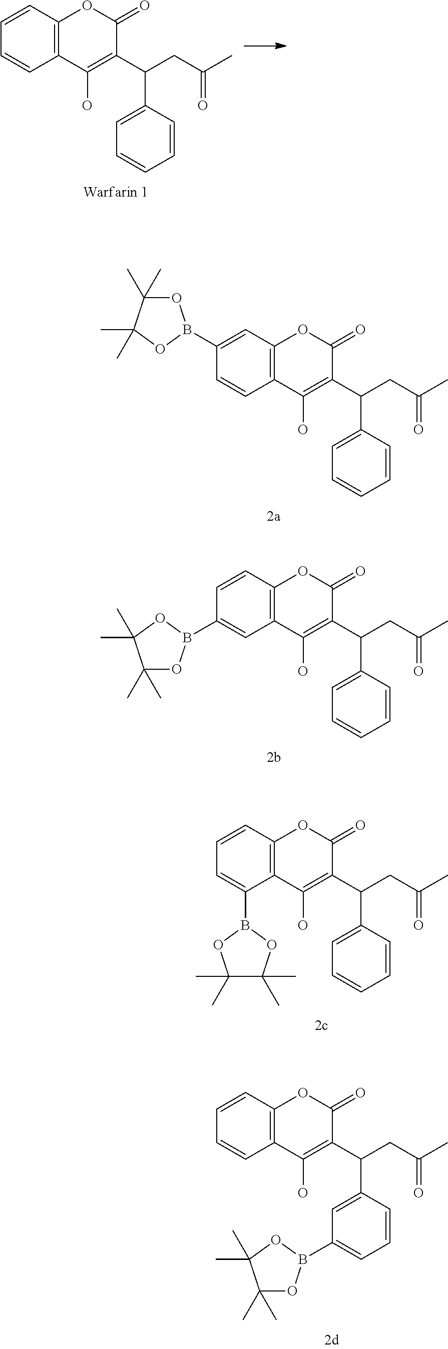

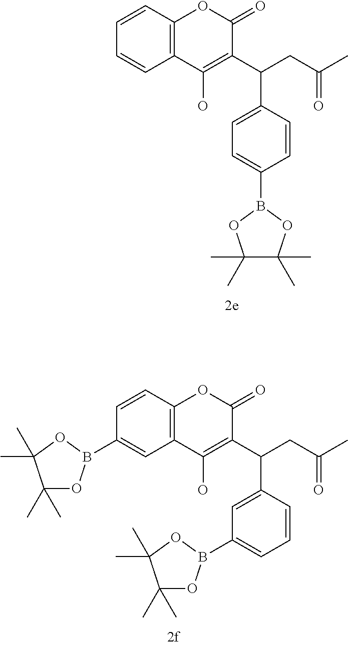

Step 1: Warfarin 1 (5 mmol) is dissolved in 2-Me-THF along with bis(pinacol)diboron (10 mmol). A solution of [(COD)IrOMe]₂ (0.125 mmol) and 3,4,7,8-tetramethyl-1,10-phenanthroline (0.25 mmol). The mixture is heated to 80° C. (suitable range is from room temperature to 130° C.) for 16 h (suitable range is 2-72 h). Upon completion of the reaction, a mixture of compounds 2a-2f along with a small amount of unreacted 1 is obtained.

Step 2: Compounds 2a-2f are separated and isolated by preparative supercritical fluid chromatography (SFC) in 10-20% isolated yield each. Chromatographic conditions: CHIRALPAK® IA™ (Chiral Technologies, Inc., West Chester, Pa.), 30 mm×250 mm, 20% MeCN/CO₂, 70 mL/min, 100 bar, 35° C., 254 nm detection, 100 mg/mL sample concentration in MeCN.

Step 3:

(Illustrated for compound 2a but would be applied to 2b-2f as well)

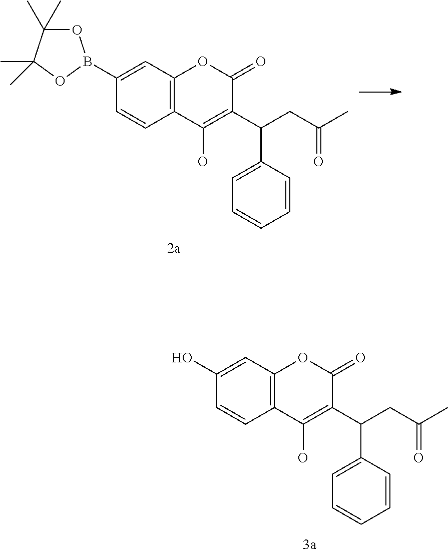 [SMILES: CC(=O)CC(c1ccccc1)c1c(O)c2ccc(B3OC(C)(C)C(C)(C)O3)cc2oc1=O ; CC(=O)CC(c1ccccc1)c1c(O)c2ccc(O)cc2oc1=O]

Compounds 2a-2f are treated with potassium peroxymonosulfate (OXONE®, 1 eq.) in acetone for 10 min at room temperature followed by 1 eq. of NaIO₄ for 1-2 h to give, after isolation, the corresponding phenols 3a-3f. See Shi et al., 2006, Org. Lett. 8, 1411-1414.

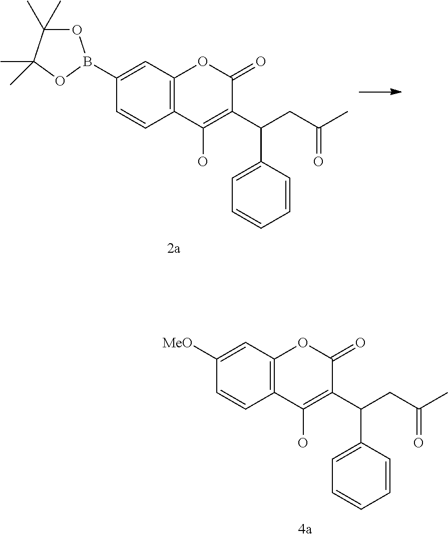 [SMILES: CC(=O)CC(c1ccccc1)c1c(O)c2ccc(B3OC(C)(C)C(C)(C)O3)cc2oc1=O ; COc1ccc2c(O)c(C(CC(C)=O)c3ccccc3)c(=O)oc2c1]

Compounds 2a-2f are treated with methanol in the presence of copper(II) acetate hydrate (10 mol %), 4-dimethylamino-pyridine (20 mol %) and 4 Å molecular sieves in dichloromethane at room temperature under an atmosphere of air for 24 h to give, after isolation, the corresponding methyl ethers 4a-4f. See Quach et al., 2003, Org. Lett. 5:1381-1384.

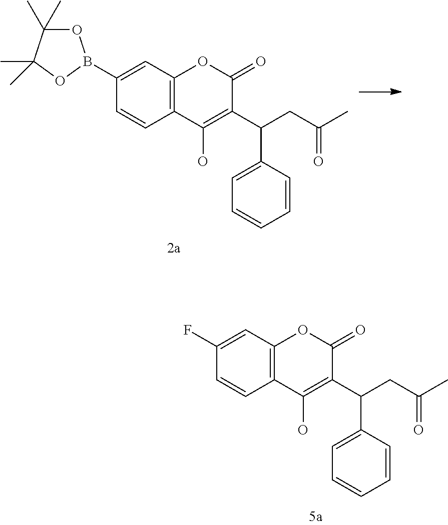 [SMILES: CC(=O)CC(c1ccccc1)c1c(O)c2ccc(B3OC(C)(C)C(C)(C)O3)cc2oc1=O ; CC(=O)CC(c1ccccc1)c1c(O)c2ccc(F)cc2oc1=O]

Compounds 2a-2f are treated with Chloromethyl-4-fluoro-1,4-diazoniabicyclo[2.2.2]octane bis(tetrafluoroborate) (SELECTFLUOR®; 1 equiv.) in acetonitrile at room temperature for 24 h to give the corresponding aryl fluoride derivatives 5a-5f. See Cazorla et al., 2009, Tetrahedron Lett. 50: 3936-3938. For alternate conditions, see: Furuya et al., 2009, Org. Lett. 11:2860-2863.

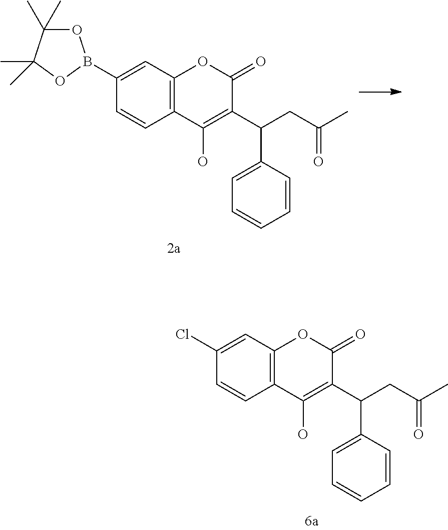 [SMILES: CC(=O)CC(c1ccccc1)c1c(O)c2ccc(B3OC(C)(C)C(C)(C)O3)cc2oc1=O ; CC(=O)CC(c1ccccc1)c1c(O)c2ccc(Cl)cc2oc1=O]

Compounds 2a-2f are treated with copper(II) chloride in aqueous methanol to give the corresponding aryl chloride derivatives 6a-6f. See Murphy et al., 2007, J. Am. Chem. Soc. 129:15434-15435. This procedure may also be used to convert the arylboronate esters 2a-2f to the corresponding aryl bromides by substituting CuBr₂ for CuCl₂.

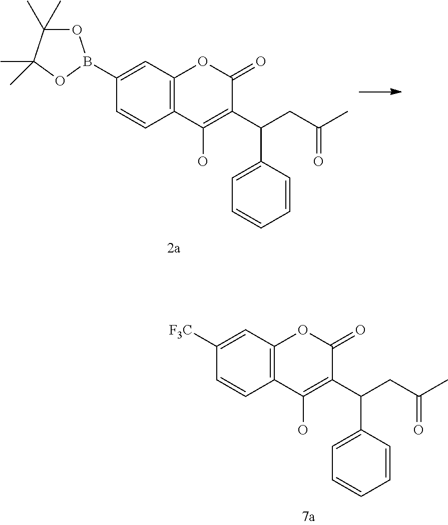 [SMILES: CC(=O)CC(c1ccccc1)c1c(O)c2ccc(B3OC(C)(C)C(C)(C)O3)cc2oc1=O ; CC(=O)CC(c1ccccc1)c1c(O)c2ccc(C(F)(F)F)cc2oc1=O]

Compounds 2a-2f are treated with copper(II) acetate (1 equiv.), phenanthroline (1.1 equiv.), cesium fluoride (2 equiv.) and (trifluoromethyl)trimethylsilane (“Rupert's Reagent”, 2 equiv.) in the presence of 4 Å molecular sieves in 1,2-dichloroethane solvent at room temperature under an air atmosphere for 16 h giving the corresponding trifluoromethyl derivatives 7a-7f. See Senecal et al., 2011, J. Org. Chem. 76:1174-1176. For alternate conditions, see: Litvinas et al., 2011, Angew. Chem. Int. Ed. 51:536-539; Chu et al., 2010, Org. Lett. 12:5060-5063; and Ye et al., 2012, J. Am. Chem. Soc. 134:9034-9037.

While the foregoing specification teaches the principles of the present invention, with examples provided for the purpose of illustration, the practice of the invention encompasses all of the usual variations, adaptations and/or modifications that come within the scope of the following claims.

1. A method for generating a compound library comprising the following steps:
a) functionalizing a compound using a C—H functionalization chemistry to form a mixture of compounds; and
b) performing one or more post-functionalization modification chemistries on the mixture of compounds.

2. The method of claim 1 further comprising testing of one or more physical or biological properties of the mixture of compounds after a) and before b), or testing of one or more physical or biological properties of the mixture of compounds after step b).

3. The method of claim 1 further comprising testing of one or more physical or biological properties of the mixture of compounds after a) and before b) and testing of one or more physical or biological properties of the mixture of compounds after step b).

4. The method of claim 1 comprising a) functionalizing a compound using a C—H functionalization chemistry to form a mixture of functionalized compounds; b) separating the mixture of functionalized compounds to obtain individual compounds; and c) performing one or more post-functionalization modification chemistries on the individual compounds.

5. The method of claim 4 further comprising testing of one or more physical or biological properties of the mixture of compounds after a) and before b); testing of one or more physical or biological properties of the individual compounds after b) and before c); or testing of one or more physical or biological properties of the individual compounds after step c).

6. The method of claim 4 further comprising testing of one or more physical or biological properties of the mixture of compounds after a) and before b); testing of one or more physical or biological properties of the individual compounds after b) and before c); and testing of one or more physical or biological properties of the individual compounds after step c).

7. The method of claim 1 comprising a) functionalizing a compound using a C—H functionalization chemistry to form a mixture of functionalized compounds; b) performing one or more post-functionalization modification chemistries on the mixture of functionalized compounds to from a mixture of modified compounds; and c) separating the mixture of modified compounds to obtain individual compounds.

8. The method of claim 7 further comprising testing of one or more physical or biological properties of the mixture of products after a) and before b); testing of one or more physical or biological properties of the mixture of compounds after b) and before c); or testing of one or more physical or biological properties of the individual compounds after step c).

9. The method of claim 7 further comprising testing of one or more physical or biological properties of the mixture of compounds after a) and before b); testing of one or more physical or biological properties of the mixture of compounds after b) and before c); and testing of one or more physical or biological properties of the individual compounds after step c).

10. The method of claim 1 wherein the compound is selected from heterocycles, steroids, alkaloids, and peptides/mimetics.

11. The method of claim 1 wherein the compound has more than two or more functionalization sites.

12. The method of claim 11 wherein the mixture comprises mono-and poly-functionalized compounds.

13. The method of claim 11 wherein one or more functionalization sites is blocked with a protecting group.

14. The method of claim 1 wherein the functionalization chemistry is selected from C—H borylation, C—H halogenation, C—H oxidation, C—H peroxidation, C—H acetoxylation, C—H amination, C—H carboxylation, C—H alkoxycarbonylation, C—H aminocarbonylation, C—H cyanation, C—H arylation, C—H nitration and C—H (fluoro)alkylation.

15-16. (canceled)

17. The method of claim 4 wherein the separating is by a chromatography method.

18. The method of claim 7 wherein the separating is by a chromatography method.

19. The method of claim 1 wherein the one or more post-functionalization modification chemistries is selected from olefination, arylation, alkylation, borylation, halogenation, carboxylation, carbonylation, amination, alkoxylation, cyanation and hydroxylation.

20-21. (canceled)

22. The method of claim 19 wherein the one or more post-functionalization modification chemistries are carried out in parallel.

23. The method of claim 2 wherein the testing is selected from mass spectrometry, liquid chromatography, and NMR.

24. The method of claim 2 wherein the testing is for a biological property.

25-28. (canceled)
